(19) United States
(12) Patent Application Publication
(10) Pub. No.: US 2015/0190613 A1
(43) Pub. Date: Jul. 9, 2015

(54) MANUALLY POSITIONED ARMATURE SYSTEM AND METHOD OF USE

(71) Applicant: STEADFAST SURGICAL, INC., La Jolla, CA (US)

(72) Inventors: David Karl Stroup, La Jolla, CA (US); Jonas Dean Cochran, La Jolla, CA (US)

(21) Appl. No.: 14/415,722

(22) PCT Filed: Jul. 10, 2013
(86) PCT No.: PCT/US2013/049930

§ 371 Date: Jan. 19, 2015

Related U.S. Application Data
(60) Provisional application No. 61/673,834, filed on Jul. 20, 2012.

(51) Int. Cl. A61M 25/01; A61B 17/02; A61B 19/00; A61B 1/00

(52) U.S. Cl. CPC A61M 25/0127; A61M 25/0155; A61B 1/00147; A61B 17/02; A61B 19/26; A61B 2017/00539

(57) ABSTRACT

A manually positionable armature system including a flexible semi-rigid housing including an inner wall, a connecter coupled to the housing to connect the housing to a holding structure, a holder coupled to the housing to hold an instrument in place during a procedure, a compressible media disposed within the flexible semi-rigid housing, an adjustable volume member disposed within the compressible media within the flexible semi-rigid housing, the adjustable volume member adjustable between at least a first volume state where the compressible media is less compressed against the inner wall of the housing and the flexible semi-rigid housing is freely positionable and a second volume state where the compressible media is more compacted against the inner wall of the housing, rigidizing and fixing the flexible semi-rigid housing in a desired orientation.

### FIELD OF THE INVENTION

This invention relates generally to positioning devices, and more particularly to armature systems, devices, and methods that permit medical instruments to be manually positioned as desired and subsequently held in place during surgical procedures.

### BACKGROUND OF THE INVENTION

Numerous fields benefit from mechanical positioning means that make it possible both to manually position objects or instruments in a chosen orientation and then rigidly hold the object or instrument, so oriented, in place. Perhaps nowhere is this ability more prized than in a surgical setting, where the ability to reliably position a surgical instrument for extended periods of time can positively influence the very outcome of the procedure.

What is desirable, but not provided by prior art systems, devices, and methods, is a means for actively rigidizing initially flexible instrument positioning means in a precise and fixed orientation.

### SUMMARY OF THE INVENTION

An aspect of the invention involves a manually positionable armature system comprising: a fixed hub; a reinforced flexible tubular housing proximally seated in said hub; a balloon axially disposed within said housing and inflatable through said hub; compressible media, comprised of regular or irregular micro solids, disposed between said balloon and said housing; and a clamp distally disposed on said housing. In a first state, said axial balloon is deflated and said housing is freely positionable. In a second state, said axial balloon is either remotely or locally hydraulically inflated so that said compressible media is compacted against the inner wall of said housing, rigidizing said armature in a desired orientation.

Another aspect of the invention involves a manually positionable armature system comprising: a flexible semi-rigid housing including an inner wall, a connecter coupled to the housing to connect the housing to a holding structure, a holder coupled to the housing to hold an instrument in place during a procedure, a compressible media disposed within the flexible semi-rigid housing, an adjustable volume member disposed within the compressible media within the flexible semi-rigid housing, the adjustable volume member adjustable between at least a first volume state where the compressible media is less compressed against the inner wall of the housing and the flexible semi-rigid housing is freely positionable and a second volume state where the compressible media is more compacted against the inner wall of the housing, rigidizing and fixing the flexible semi-rigid housing in a desired orientation.

In one embodiment, the space between balloon and housing is separated into successive chambers by spaced annular baffles extending between the balloon and housing. In this embodiment, portions of the balloon in each chamber are inflated to compact media in the respective chambers against the inner wall of the housing. The baffles help to keep the balloon centered in the housing and also provide more even radial distribution of compressible media along the length of the housing. In one embodiment, a semi-rigid tubular support member or manifold extends inside the balloon and has openings along its length through which pressurized inflation fluid flows to inflate or deflate successive portions of the balloon in each chamber.

### BRIEF DESCRIPTION OF THE DRAWINGS

FIG. 1A is a cross-sectional side view of a first embodiment of a positionable armature device;

FIG. 1B is a perspective view of the positionable armature device of FIG. 1A;

FIG. 2A is a cross-sectional side view showing an adjustable volume member and compressible media of the positionable armature device of FIG. 1A in a first state;

FIG. 2B is an enlarged cross-sectional view of the circled area of FIG. 2A showing a first embodiment of an adjustable volume member and compressible media of the positionable armature device of FIG. 1A in a first state;

FIG. 3A is a cross-sectional side view showing an adjustable volume member and compressible media of the positionable armature device of FIG. 1A in a second state;

FIG. 3B is an enlarged cross-sectional view of the circled area of FIG. 3A showing an adjustable volume member and compressible media of the positionable armature device of FIG. 1A in a second state;

FIG. 4A is a cross-sectional side view similar to FIG. 2A illustrating a second embodiment of a positionable armature device in a first, straight condition;

FIG. 4B is an enlarged cross-sectional view of the circled area of FIG. 4A showing an adjustable volume member and compressible media of the positionable armature device in a first state;

FIG. 5A is a cross-sectional view on the lines 5A-5A of FIG. 4B;

FIG. 5B is a cross-sectional view on the lines 5B-5B of FIG. 4B;

FIG. 6A is a cross-sectional side view similar to FIG. 4A showing the adjustable volume member and compressible media in a second, curved condition;

FIG. 6B is an enlarged cross-sectional view of the circled area of FIG. 6A;

FIG. 7A is a cross-sectional side view similar to FIG. 4A showing the adjustable volume member of the adjustable volume assembly and the compressible media in an expanded condition and a curved configuration;

FIG. 7B is an enlarged cross-sectional view of the circled area of FIG. 7A;

FIG. 8 is an enlarged cross-sectional view similar to FIG. 7B but with the positionable armature device in a straight configuration; and

FIG. 9 is a cross-sectional view on the lines 9-9 of FIG. 8.

### DETAILED DESCRIPTION OF THE PREFERRED EMBODIMENTS

Certain embodiments provide a system and method for manually positioning and rigidizing an armature device.

The following detailed description is directed to embodiments of the invention in which a manually positionable armature system permit medical instruments (e.g., endoscopes, retractors, etc.) to be manually positioned as desired and subsequently held in place during surgical procedures. However, the invention can be embodied in a multitude of different ways as defined and covered by the claims and may be used to hold in place instruments other than medical instruments in applications in non-medical/surgical procedures. In this description, reference is made to the drawings wherein like parts are designated with like numerals throughout.

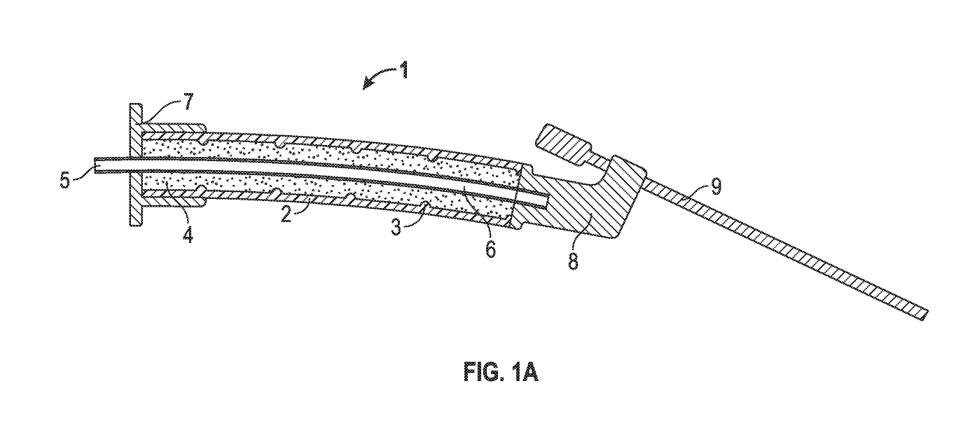

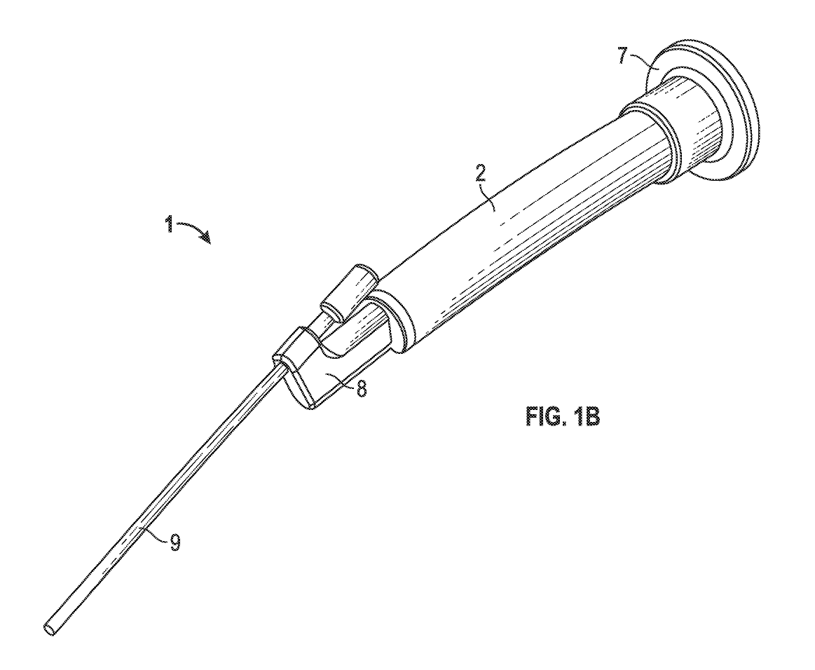

FIGS. 1A to 3B illustrate a first embodiment of a manually positionable armature device 1 for holding an instrument 9. As best illustrated in FIGS. 1A and 1B, manually positionable armature device 1 of this embodiment includes a holder in the form of a clamp 8 fixedly attached to the distal end of flexible semi-rigid housing 2. The clamps 8 holds an instrument 9 (e.g., medical/surgical instrument) in place during a procedure (e.g., medical/surgical procedure).

The proximal end of flexible housing 2 is mechanically fitted to fixed hub 7. The hub 7 (or a connecter coupled to the hub) connects the housing 2 to a holding structure, which may be stationary or moveable.

An adjustable volume member in the form of an axial balloon 6 is centrally disposed within the compressible media 4 in the flexible housing 2. A distal end of axial balloon 6 is fixedly attached to clamp 8. A proximal end of the balloon 6 is fixedly attached to inlet/outlet tube 5, which extends through fixed hub 7 and is operatively connected to a fluid pressure source (not illustrated). The fluid pressure source is preferably a hydraulic pump/cylinder, but in alternative embodiments, the fluid pressure source may be an air cylinder with water, another type of pump, or another type of fluid pressure source.

The flexible semi-rigid housing 2 includes an inner wall with internal features or annular ribs 3 sized and shaped to maintain axial balloon 6 centrally disposed in the compressible media within the housing 2 as shown. Without internal features 3, the balloon 6 would tend to move towards the inner wall of the housing 2 in one or more locations of bending. The flexible semi-rigid housing 2 does not allow for radial or longitudinal expansion of the housing 2 (i.e., housing has fixed radius/diameter in all states of axial balloon) under load.

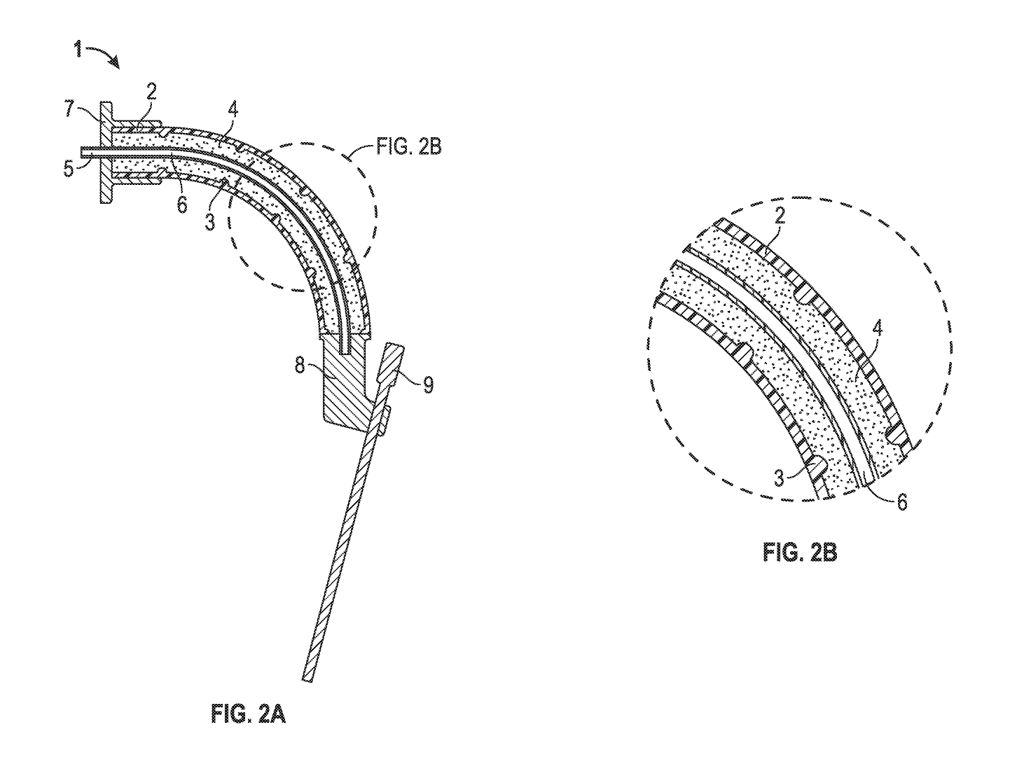

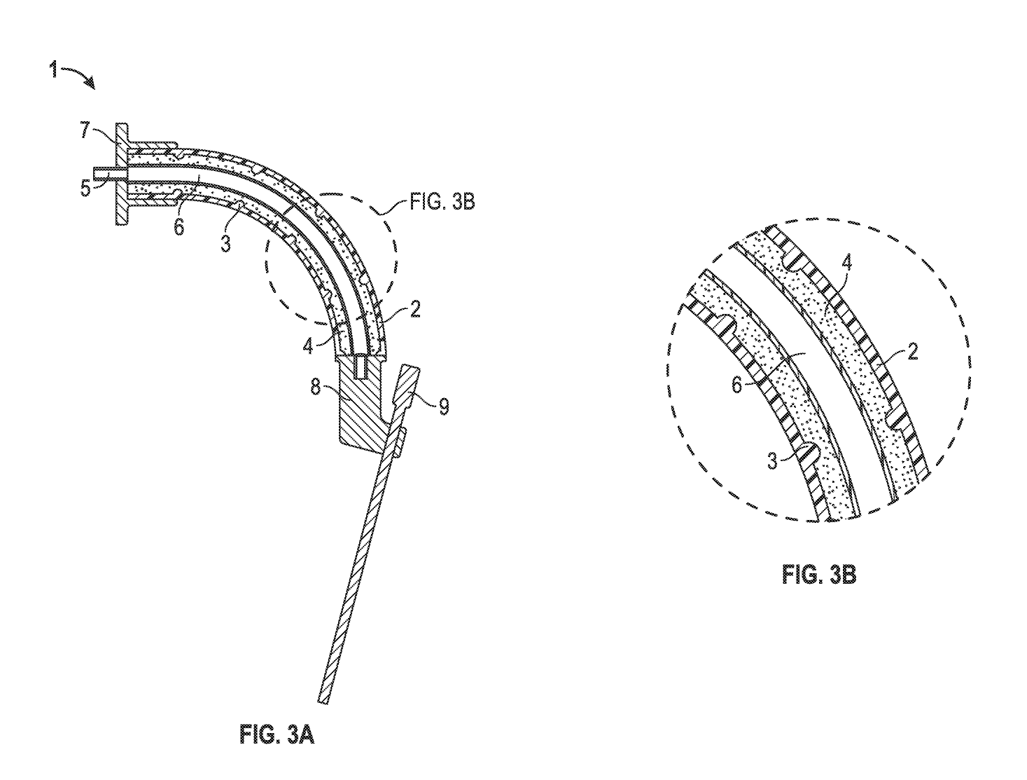

Compressible material 4 is disposed within flexible housing 2 and can have physical properties resembling either sand (i.e., irregularly shaped micro solids) or glass bead blasting material (i.e., regular shaped micro solids). Compressible material 4 is distributed in sufficient quantity to allow housing 2 to achieve a desired rigidity when axial balloon 6 is inflated via pump 10 (FIGS. 3A, 3B) and yet also allow flexible housing 2 to be positioned or repositioned when axial balloon 2 is deflated (FIGS. 2A, 2B).

As shown in FIGS. 3A and 3B, axial balloon 6, which may have elastomeric or non-elastomeric physical properties, is inflated relative to that shown in FIGS. 1A & 1B, FIGS. 2A & 2B in a second state. As inflation pressures provided by the pump to axial balloon 6 needed to achieve optimal rigidity in flexible housing 2 may exceed 150 p.s.i., it is essential that housing 2 not exhibit radial or longitudinal expansion under load. Flexible housing 2 must be both flexible enough to allow sufficient degrees of freedom for positioning device 1 when balloon 6 is deflated but also rigid enough and adequately reinforced to keep balloon 6 from having to be inflated until it reaches the elastic limit of housing 2. In practice, this necessitates housing 2 having a minimum burst pressure of about 1,000 p.s.i. The outer housing or hose may be of any suitable flexible reinforced material. Suitable materials for housing 2 include hydraulic hose of rubber, synthetic rubber, nylon, polyurethane, or the like, reinforced with braided steel wire or fiber braid of a material such as polyester, aramid, or rayon.

In the above arrangement, not only do internal features 3 of flexible housing 2 center the axial balloon 6, but also serve as reactive surfaces against the pressure of compressible material 4 in a compressed state, the condition achieved when axial balloon 6 is inflated. In this state, compressible material 4 behaves like a solid; that is, it is unable to move or flow within housing 2, thereby providing a desired rigidity in device 1.

In an implementation where the fluid pressure source is a hydraulic pump, the hydraulic fluid used in the pump to inflate balloon 6 is preferably either mineral oil or silicone oil.

In use, the manually positionable armature device 1 is connected to a holding structure via the hub 7 or a connecter coupled to the hub 7. At an opposite end, the instrument 9 (e.g., medical/surgical instrument) is held by the manually positionable armature device 1 via clamp 8. In the first, non-inflated state of the balloon 6 as shown in FIGS. 2A, 2B where the compressible media is not compacted against the inner wall of the housing 2, the housing 2 is flexible/movable and the manually positionable armature device 1 is manually positioned to a desired orientation/configuration for the procedure/instrument 9. When the armature device 1 is manually positioned to the desired orientation/configuration for the procedure, the fluid pressure source is actuated, pumping fluid (e.g., water) into the balloon 6. This causes the balloon 6 to expand within the compressible media 4 to the second, inflated state shown in FIGS. 3A, 3B. In this state, the compressible media 4 is compacted against the inner wall of the semi-rigid housing 2, which does not allow for radial or longitudinal expansion under load. This causes the semi-rigid housing 2, and, hence, the manually positionable armature device 1, to rigidize into the desired orientation/configuration for the procedure.

With the manually positionable armature device 1 of the present invention, the manually positionable armature device 1 is able to achieve much greater rigidity than in manually positionable armature devices in the past, in part, because the balloon 6 is centrally disposed in the semi-rigid housing and pushes outward, when inflated, to compact the compressible media 4 against the inner wall of the semi-rigid housing 2, the semi-rigid housing 2 does not expand (radially or longitudinally), and the balloon 6 is capable of achieving much greater pressures or driving forces (e.g., rated 1000-2000 psi).

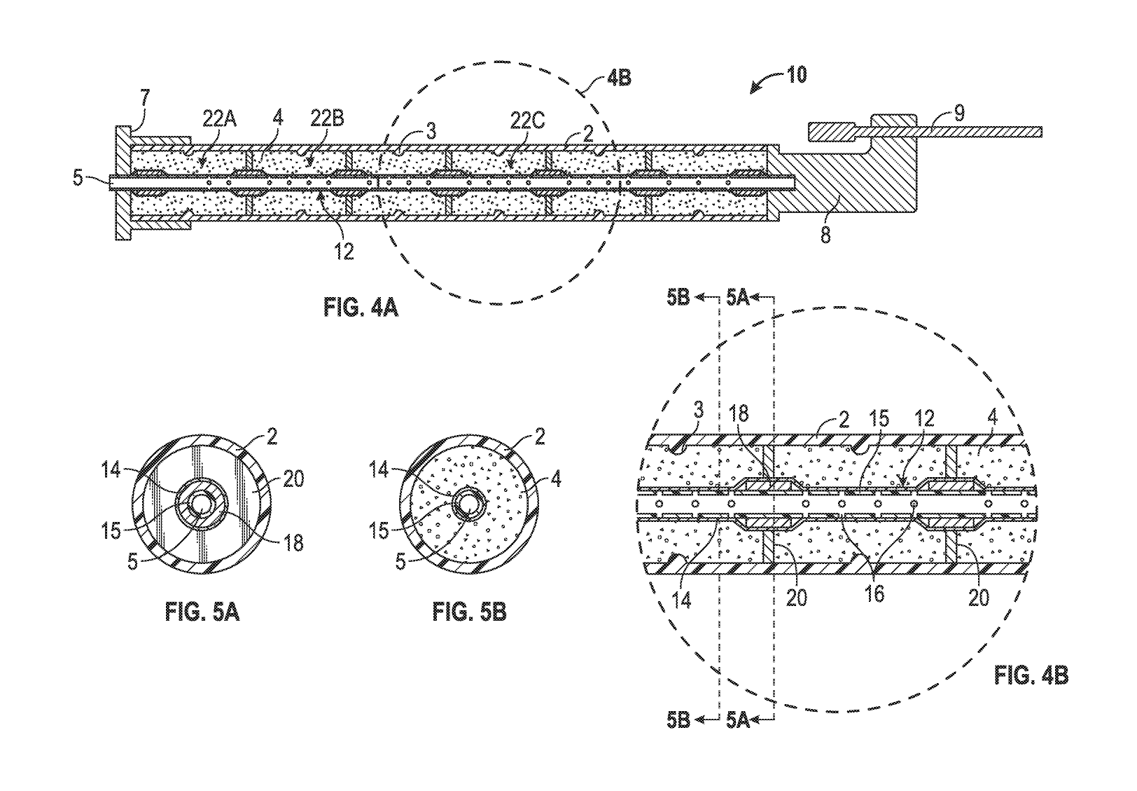

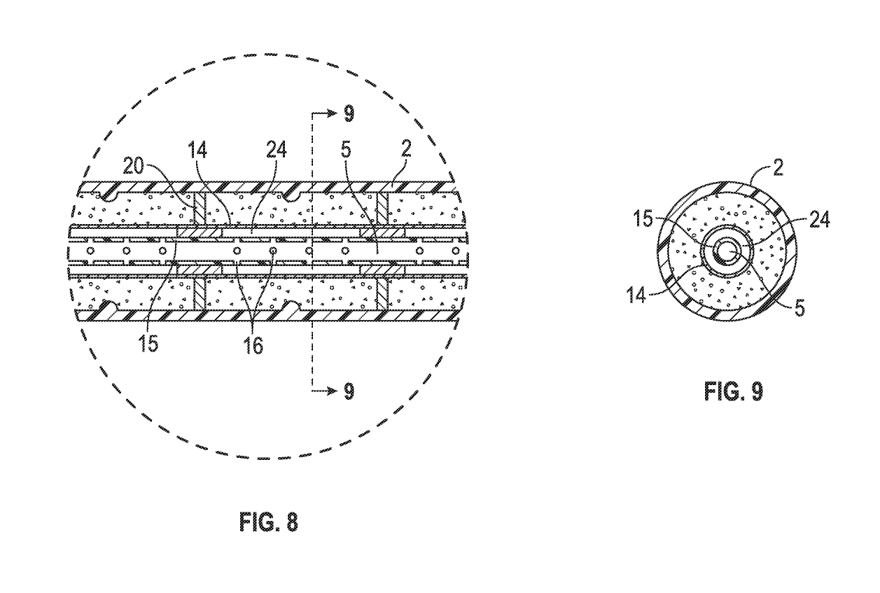

FIGS. 4A to 9 illustrate a second embodiment of a manually positionable armature device 10 which has a modified adjustable volume assembly 12 including adjustable volume member or axially extending balloon 14 as well as spaced annular baffles 20 between the adjustable volume assembly 12 and the housing 2. The device 10 is otherwise similar or identical to the first embodiment, and like reference numerals are used for like parts as appropriate.

As in the first embodiment, manually positionable armature device 10 includes a holder 8 in the form of a clamp fixedly attached to the distal end of flexible, semi-rigid tubular housing 2, and a hub 7 mechanically fitted to the proximal end of housing 2. Clamp 8 holds an instrument 9, which may be a medical or surgical instrument, in a desired orientation during a procedure. As in the previous embodiment, housing 2 has internal features 3 such as spaced annular ribs or projections, and is not expandable either radially or longitudinally, although it may be bent to a desired orientation and then locked in that orientation, as described in more detail below.

Tubular adjustable volume assembly 12 extends axially through the center of housing 2 and compressible media 4 fills the annular space between assembly 12 and the inner surface of housing 2. Media 4 may be similar or identical to that described above in connection with the first embodiment. As best illustrated in FIGS. 4B, 5A and 5B, the adjustable volume balloon is supported by a tubular, semi-rigid manifold 15 which extends inside the axially extending adjustable volume member or axially extending balloon 14, and supports the balloon in the deflated condition of FIGS. 4A to 6B. The manifold may be of any suitable unreinforced semi-rigid material such as polyurethane or polyethylene. Rigid annular spacers or supports 18 are provided at spaced intervals between semi-rigid manifold 15 and balloon 14, and the annular baffles 20 extend between the adjustable volume assembly and the housing 2 at respective supports 18, separating the interior into separate annular chambers 22A, 22B, 22C, etc. between the baffles which each contain compressible media 4.

The semi-rigid manifold 15 has plural openings 16 along its length, and may be made of any suitable semi-rigid material which will allow bending of the overall structure for adjustment purposes. In one embodiment, manifold 15 and outer housing 2 may be of the same materials. With this arrangement, the device can still be bent or curved to the desired orientation for positioning instrument 9 when in its non-expanded state, but the manifold 15 and outer tube or housing 2 do not expand radially or longitudinally when inflating fluid is supplied to tubular passageway 5 inside manifold 15. The baffles 20 perform two functions. First, they keep the manifold 15 and balloon 14 centered in outer housing 2, to provide an even radial distribution of media around balloon 14 during expansion and contraction. Second, they retain the media in separate annular chamber portions to even out the distribution of media longitudinally in the device during repeated expansion and contraction.

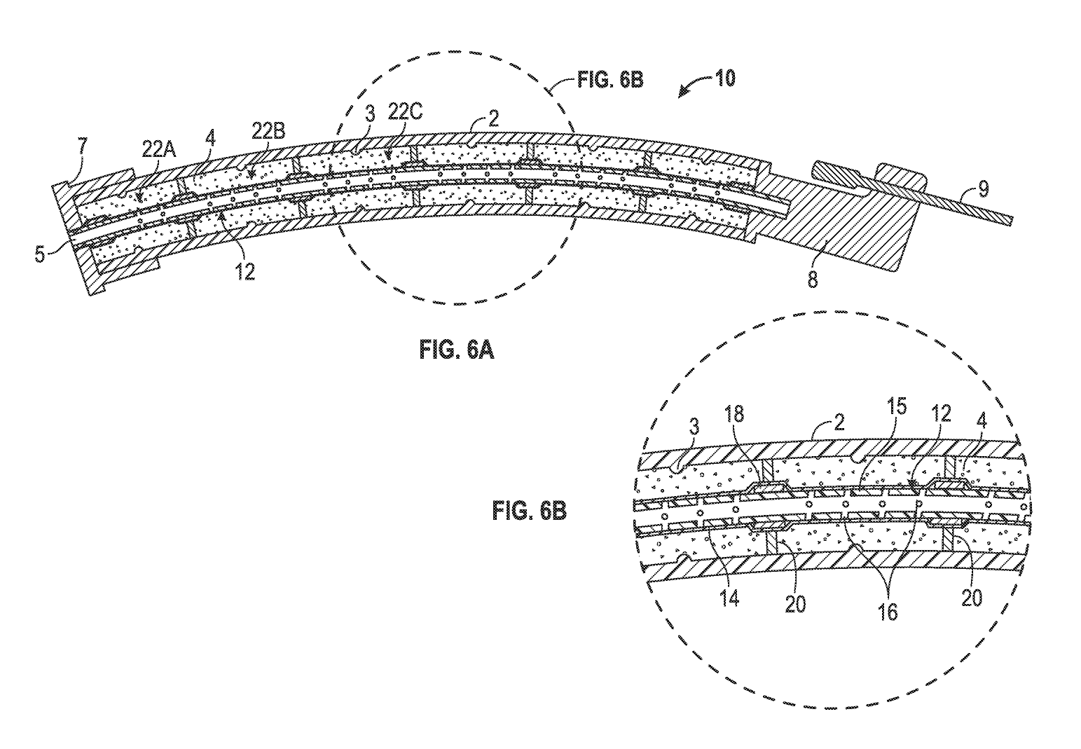

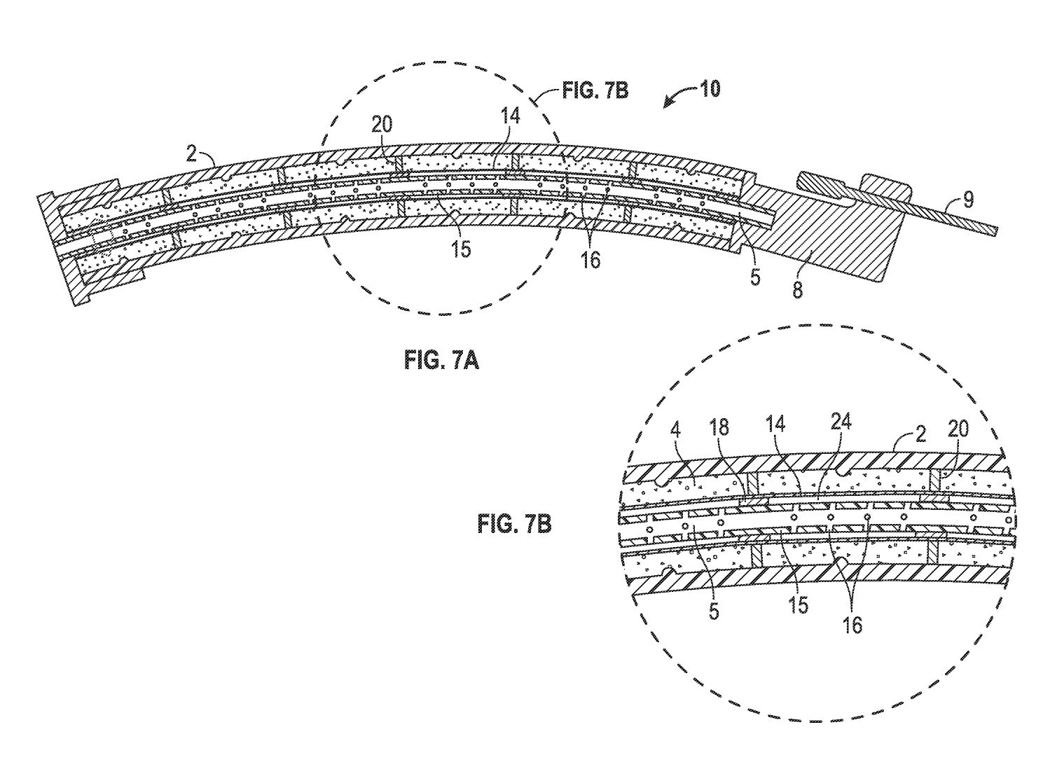

FIGS. 4A and 4B illustrate the device 10 in a straight or linear orientation with balloon 14 in a first, non-inflated state or deflated, while FIGS. 6A and 6B illustrate one possible bent or curved orientation of device 10, also in the non-inflated state. Clearly, housing 2 can be manually bent into other orientations with greater or lesser curvature when in the non-inflated state, depending on the desired orientation or position of instrument 9 relative to the holder or mount on which the proximal end of device 10 is mounted via hub 7. When the desired orientation (either straight or curved) is reached for the procedure, the fluid pressure source is actuated, pumping pressurized fluid (e.g., water) along tubular passageway 5. The fluid flows outwardly through openings 16 in manifold 15 into the spaces between manifold 15 and balloon 14, causing the portions of balloon 14 between baffles 20 to expand within the compressible media 4 to the second, inflated state shown in FIGS. 7A to 9 in which there is an annular, fluid-filled space 24 between the manifold 15 and expanded balloon section between each pair of baffles 20. In this state, the compressible media 4 in each chamber 22 is compacted against the inner wall of the semi-rigid housing 2, which does not allow for radial or longitudinal expansion under load. This causes the semi-rigid housing 2, and, hence, the manually positionable armature device 1, to rigidize into the desired orientation/configuration for the procedure.

When the procedure is completed, fluid may be evacuated from the device so that the balloon deflates back to the non-inflated condition of FIGS. 4A to 6B. In this embodiment, the semi-rigid inner manifold, spacers 18, and baffles 20 serve to keep the balloon centered in the outer housing 2 and to keep the media 4 substantially evenly distributed both radially and longitudinally around the balloon. This allows the balloon to expand and collapse more evenly.

With the manually positionable armature device 1, 10 of the above embodiments, the housing 2 is able to achieve much greater rigidity than in manually positionable armature devices in the past, in part, because the balloon is centrally disposed in the semi-rigid housing and pushes outward, when inflated, to compact the compressible media against the inner wall of the semi-rigid housing 2, while the semi-rigid housing 2 does not expand (radially or longitudinally), and the balloon is capable of achieving much greater pressures or driving forces (e.g., rated 1000-2000 psi).

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

1. A manually positionable armature device, comprising:
an elongated, flexible semi-rigid housing having a proximal end, a distal end and an inner wall defining a chamber within the housing extending between said proximal and distal ends;
a connector at the proximal end of the housing configured for connecting the housing to a holding structure;
an instrument holder connected to the distal end of the housing and configured to hold an instrument in place during a procedure;
an adjustable volume member disposed within the housing chamber and spaced from said inner wall, the adjustable volume member being movable between a first volume state and a second, expanded volume state;
a compressible medium disposed within the housing chamber between said adjustable volume member and said inner wall of the housing, the compressible medium being flowable when the adjustable volume member is in the first volume state and substantially non-flowable when the adjustable volume member is in the second volume state; and
the flexible semi-rigid housing being manually bendable between straight and curved orientations when the adjustable volume member is in the first volume state to adjust the position and orientation of said instrument holder at the distal end of the housing, and being fixed in a selected orientation when the adjustable volume member is in the second, expanded volume state.

2. The device of claim 1, wherein the adjustable volume member comprises a balloon and an inlet/outlet tube connected to the balloon and extending out of the housing, the inlet/outlet tube being configured for connection to a fluid pressure source to control inflation and deflation of the balloon.

3. The device of claim 2, wherein the balloon extends between the proximal and distal ends of the housing.

4. The device of claim 3, wherein the balloon is tubular in shape and has a first diameter in the first volume state and a second, larger diameter in the second, expanded volume state.

5. The device of claim 1, wherein the housing is tubular and has at least substantially the same diameter in the first and second volume states of the expandable member.

6. The device of claim 5, wherein the compressible medium comprises a plurality of solid particles which are flowable relative to one another when the expandable member is in the first volume state and which are substantially non-flowable and compressed against the inner wall of the housing when the expandable member is in the second, expanded volume state.

7. The device of claim 6, wherein the compressible medium is glass bead blasting material.

8. The device of claim 6, wherein the compressible medium is sand-like.

9. The device of claim 1, wherein the inner wall of the housing has internal reinforcing projections.

10. The device of claim 9, wherein the projections comprise spaced annular ribs.

11. The device of claim 1, wherein the adjustable volume member is an elongate balloon member extending between the proximal and distal ends of the housing and having a first diameter in the non-expanded state.

12. The device of claim 11, further comprising a tubular support member of semi-rigid material extending coaxially within said balloon member and having an outer diameter substantially equal to the first diameter of the balloon member in the first volume state, the tubular support member having a plurality of openings along its length and a proximal end extending out of the housing, the proximal end being configured for connection to a fluid pressure source to control inflation and deflation of the balloon member, whereby pressurized fluid flows through the tubular support member and out through the openings to inflate the balloon member.

13. The device of claim 12, further comprising a plurality of annular baffles extending radially at spaced intervals in the chamber between the support and balloon members and the housing, the baffles dividing the chamber into a series of separate annular chambers along the length of the housing.

14. The device of claim 13, wherein the baffles are of at least substantially rigid material.

15. The device of claim 13, further comprising a plurality of tubular spacers between the support member and balloon member at spacer positions located at spaced intervals along the length of the tubular support member.

16. The device of claim 15, wherein each baffle is located between the support and balloon members at a respective spacer position.

17. The device of claim 13, wherein the expanded state of the balloon member comprises a series of expanded balloon portions radially spaced from the tubular support member, each expanded balloon portion being located between respective adjacent pairs of baffles in a respective annular chamber.

18. A manually positionable armature system, comprising:
a fixed hub;
a reinforced flexible tubular housing proximally seated in said hub and having an inner wall;
a balloon axially disposed within said housing and inflatable through said hub;
a compressible medium including regular or irregular micro solids, the compressible medium disposed between said balloon and the inner wall of said housing; and
a clamp distally disposed on said housing,
wherein, in a first state, said axial balloon is deflated and said housing is freely positionable, and, in a second state, said axial balloon is either remotely or locally hydraulically inflated so that said compressible media is compacted against the inner wall of said housing, rigidizing said armature in a desired orientation.

19. The system of claim 18, further comprising a tubular support member axially disposed within said balloon and supporting said axial balloon along its length in the first state.

20. The system of claim 19, wherein the tubular support member has a plurality of openings along its length and has a proximal end configured for connection to a fluid pressure source to control inflation and deflation of the balloon through said openings.

21. The system of claim 20, further comprising a plurality of annular baffles at spaced intervals along the length of the tubular support member and balloon, each annular baffle extending radially between the balloon and inner wall of said housing to form a plurality of separate chambers containing compressible medium along the length of the housing, whereby portions of said axial balloon between adjacent baffles are inflated in said second state so that said compressible medium in each chamber is compacted against the inner wall of said housing, rigidizing said armature in a desired orientation

22. A method of manually positioning an instrument and holding the instrument in the selected position, comprising:
connecting a proximal end of a flexible semi-rigid tubular housing to a holding structure;
securing an instrument to a clamp located at a distal end of the tubular housing:
manually bending the tubular housing into a selected configuration in which the instrument is in a desired position and orientation for use; and
inflating a balloon extending axially within the tubular housing until a compressible material disposed in the housing between the balloon and the inner wall is sufficiently compacted against the inner wall to hold the tubular member rigidly in the selected configuration, whereby the instrument is held in the desired position.

23. The method of claim 22, wherein the step of inflating the balloon comprises inflating successive portions of the balloon in separate chambers formed between radial baffles between the balloon and inner wall of the housing until compressible material in each chamber is compacted against the inner wall to hold the tubular member rigidly in the selected configuration.
